(19) United States
(12) Patent Application Publication
(10) Pub. No.: US 2014/0066379 A1
(43) Pub. Date: Mar. 6, 2014

(54) RECOMBINANT VECTOR, TRANSGENIC FISH EGG USING THE SAME AND BIOMATERIAL USING THE SAME

(71) Applicant: Body Organ Biomedical Corp., Taipei City (TW)

(72) Inventors: Chuian-Fu Ken, Taipei City (TW); Hong-Yi Gong, Taipei City (TW); Jen-Leih Wu, Taipei City (TW); Horng-Ji Lai, Taipei City (TW); Chien-Cheng Lin, Taipei City (TW); Yong-Guei Chen, Taipei City (TW); Cheng-Hung Chou, Taipei City (TW)

(73) Assignee: Body Organ Biomedical Corp., Taipei City (TW)

(21) Appl. No.: 14/012,009

(22) Filed: Aug. 28, 2013

Related U.S. Application Data
(60) Provisional application No. 61/694,877, filed on Aug. 30, 2012.

(51) Int. Cl. C07K 14/78

(52) U.S. Cl. CPC C07K 14/78
USPC 514/17.2; 530/356; 435/325; 435/320.1

(57) ABSTRACT

A recombinant vector, transgenic fish egg using the same and biomaterial using the same are applied to provide a transgenic fish that secreting recombinant human procollagens or collagens, and further to provide the biomaterial having the recombinant human procollagens or collagens and extract the recombinant human procollagens or collagens from the part(s), having the recombinant human procollagens or collagens, of the transgenic fish.

### CROSS-REFERENCES TO RELATED APPLICATIONS

This application claims priority from U.S. Provisional Patent Application Ser. No. 61/694,877, filed on Aug. 30, 2012, which application is incorporated by reference herein.

### BACKGROUND

1. Technical Field

The invention relates to a manufacturing technology of a biomedical material, and particularly to a recombinant vector, a transgenic fish using the same and a biomaterial using the same.

2. Related Art

Collagens are important reconstruction materials in biomedical material applications, and have the capabilities of improving tissue growing, improving wound healing, using as plastic materials, using as wound dressing for burns, and etc. Additionally, the market scale of collagens continues to expand. Consequently, development of collagens and derivatives thereof with higher medical values and wider diversities is a topic for researchers to resolve.

So far, the expression system for producing recombinant collagens includes Escherichia coli (E. coli), yeast (such as Saccharomyces cerevisiae), mammalian cell lines, insect cell lines, transgenic animals and transgenic plants. The post-translational modification cannot be achieved in the E. coli expression system. The yeast expression system lacks of the activity of pmlyl-4-hygroxylase (PH), while the yeast expression system where the yeasts grown on Methanol has the best yields of the collagens among these expression systems. The mammalian cell expression system has low yields of the collagens and is limited to use particular cells. In the insect cell expression system, there is low activity of pmlyl-4-hygroxylase. For the transgenic animals (such as silk worms and mice), and transgenic plants (such as tobacco), the collagen products are over cross-linking.

Until now, baculovirus expression system with human pmlyl-4-hygroxylase and collagen genes has been established (referring to U.S. Pat. No. 5,077,214 and U.S. Pat. No. 5,593,859). However, the transfected insect cells will die within 72 hours after transfection, so that the recombinant collagens are produced temporarily. In addition, due to the cell lysis, the recombinant collagens are difficult to recycle or purify hardly. Furthermore, the damages of endoplasmic reticulum and Golgi bodies by the baculovirus further restrict collagen production.

### SUMMARY

In one embodiment, a biomaterial includes a recombinant human procollagen or collagen obtained from and secreted by a fish.

In some embodiment, the biomaterial further includes at least one fish scale of the fish, and each fish scale has the recombinant human procollagen or collagen.

In some embodiment, the recombinant human procollagen or collagen is expressed by at least one human procollagen or collagen gene in the fish.

In another embodiment, a transgenic fish egg or embryo includes a fish chromosome and a recombinant vector. The recombinant vector is inserted in the fish chromosome, and the recombinant vector has a human procollagen or collagen gene.

In yet another embodiment, a recombinant vector includes an upstream gene and a human procollagen or collagen gene. The upstream gene is constructed from a fish, and the human procollagen or collagen gene is connected to downstream of the upstream gene.

In some embodiments, the upstream gene is a promoter already existing in the fish, an enhancer already existing in the fish, or the combination thereof.

In some embodiments, the recombinant vector further includes a report gene, and the report gene is connected to downstream of the human procollagen or collagen gene.

As above, the recombinant vector, the transgenic fish using the same and the biomaterial using the same according to the invention can be applied to provide a transgenic fish that secrets the recombinant human procollagens or collagens in part(s) of the transgenic fish, and further the part(s) of the transgenic fish that secrets the recombinant human procollagens or collagens can be treated as the biomaterials and/or can be treated to purify the recombinant human procollagens or collagens.

The detailed features and advantages of the disclosure are described below in great detail through the following embodiments, the content of the detailed description is sufficient for those skilled in the art to understand the technical content of the disclosure and to implement the disclosure there accordingly. Based upon the content of the specification, the claims, and the drawings, those skilled in the art can easily understand the relevant objectives and advantages of the disclosure.

### BRIEF DESCRIPTION OF THE DRAWINGS

The disclosure will become more fully understood from the detailed description given herein below for illustration only and thus not limitative of the disclosure, wherein:

FIG. 1 is a flow chart of one embodiment of a transfect method for a transgenic fish egg;

FIG. 2 is a schematic view of one embodiment of a recombinant vector;

FIG. 3 is a detailed flow chart of one embodiment of step S10 shown in FIG. 1;

FIG. 4 is a detailed flow chart of one embodiment of step S110 shown in FIG. 3;

FIG. 5 is a detailed flow chart of one embodiment of step S120 shown in FIG. 3;

FIG. 6 is a detailed flow chart of one embodiment of step S130 shown in FIG. 3;

FIG. 7 is a detailed flow chart of one embodiment of step S140 shown in FIG. 3; and

FIG. 8 is a schematic view of one embodiment of a recombinant plasmid having human type I, alpha 1 procollagen or collagen gene.

### DETAILED DESCRIPTION

The ordinal terms, such as first, second, third, and etc., mentioned hereafter are applied to distinguish the elements only, but not applied to sequence or to restrict the elements; also they are not applied to limit the scope of the disclosure.

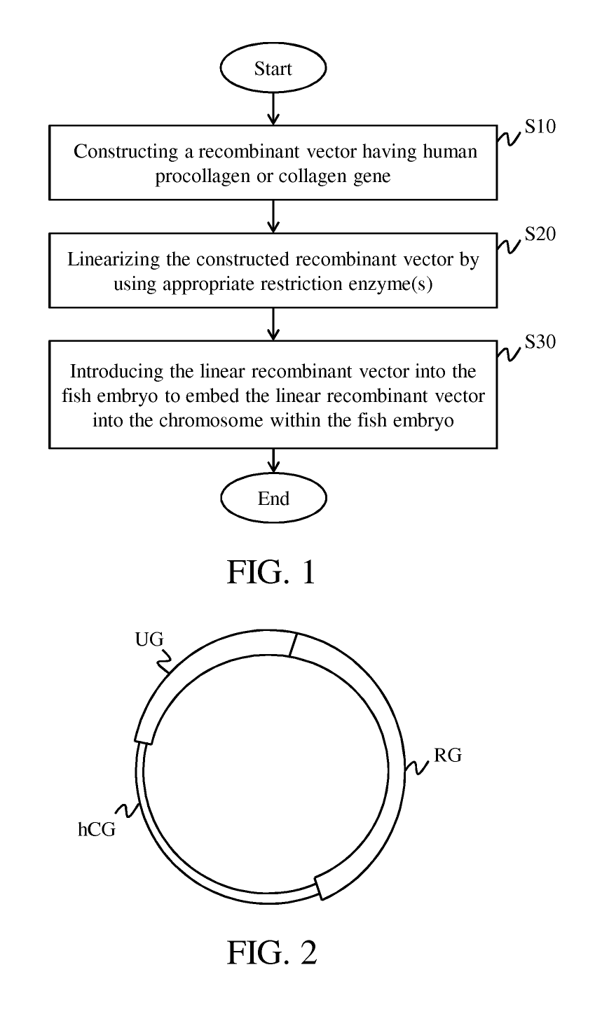

Please refer to FIG. 1, in which a method for preparing a transgenic fish egg or embryo includes constructing a recombinant vector having a human procollagen or collagen gene hCG (step S10), linearizing the constructed recombinant vector by using appropriate restriction enzyme(s) (step S20) and introducing the linear recombinant vector into the fish embryo to embed the linear recombinant vector into the chromosome within the fish embryo (step S30).

Please refer to FIG. 2, in which the recombinant vector includes an upstream gene UG and the human procollagen or collagen gene hCG, and the human procollagen or collagen gene hCG is connected to downstream of the upstream gene UG. The upstream gene UG at least has a promoter which specifically drives the expression of downstream generic sequence (that is, human procollagen or collagen gene hCG) in a germ cell (that is, embryo), an enhancer co-working with the promoter, or the combination thereof. The promoter is present in the fish and has tissue-specific. The enhancer is present in the fish.

In some embodiments, the promoter can be, for example, keratin 4 (K4) promoter, keratin 5 promoter or krtt1c6 promoter.

Wherein, the sequence of the keratin 4 promoter is shown as SEQ ID NO: 1, and the sequence of the krtt1c6 promoter is shown as SEQ ID NO: 2.

In some embodiments, the human procollagen or collagen gene hCG can be a single-stranded amino acid sequence or a triple-stranded amino acid sequence.

The human procollagen or collagen gene hCG can be one of the human type I-XXVIII procollagens or collagens or the combination formed with at least two of the human type I-XXVIII procollagens or collagens. The human procollagen or collagen gene hCG can be single-stranded or multi-stranded. For example, the human type I procollagen or collagen has two α1 chains, and one α2 chain, and the human type II procollagen or collagen has three α1 chains. As the matched types of the human procollagen or collagen are well known by those skilled in the art, additional details relating to the matched types of the human procollagen or collagen are not provided.

The sequence of the human type I procollagen or collagen gene is shown as SEQ ID NO: 3 and 4, and the sequence of the human type II procollagen or collagen gene is shown as SEQ ID NO: 5. Here, the gene sequence of each chain of the human type I and II procollagens or collagens is exemplarily shown in sequence listing, but not limited thereto.

In some embodiments, the recombinant vector can further have a report gene RG, and the report gene RG is connected to downstream of the human procollagen or collagen gene hCG.

The report gene RG can be, for example, a luciferase gene, a fluorescent protein gene, a lac Z gene (aβ-galactosidase gene) or etc.

The fluorescent protein gene can be the green fluorescent protein (GFP) gene, the modified green fluorescent protein gene, the enhanced green fluorescent protein (EGFP) gene, the red fluorescent protein (RFP) gene, the enhanced red fluorescent protein (ERFP) gene, the blue fluorescent protein (BFP) gene, the enhanced blue fluorescent protein (EBFP) gene, the yellow fluorescent protein (YFP) gene, the enhanced yellow fluorescent protein (EYFP) gene, the cyan fluorescent protein (CFP) gene, the enhanced cyan fluorescent protein (ECFP) gene, or etc.

In some embodiments, the recombinant vector can be a recombinant plasmid, a recombinant viral vector, a cosmid, or an artificial chromosome.

In some embodiments, the introducing step (the step S30) can be implemented by microinjection, transfection (such as DEAE-dextran precipitation or calcium phosphate precipitation), being infected with viral vectors (such as retrovirus or adenovirus), electroporation, increasing the cell membrane permeability, gene gun, liposome delivery (such as Lipofectin®, Lipofectamine®, Superfect®, Transfectam® or other liposome developed according to conventional methods), receptor mediated absorption, other pinocytosis mechanisms or etc.

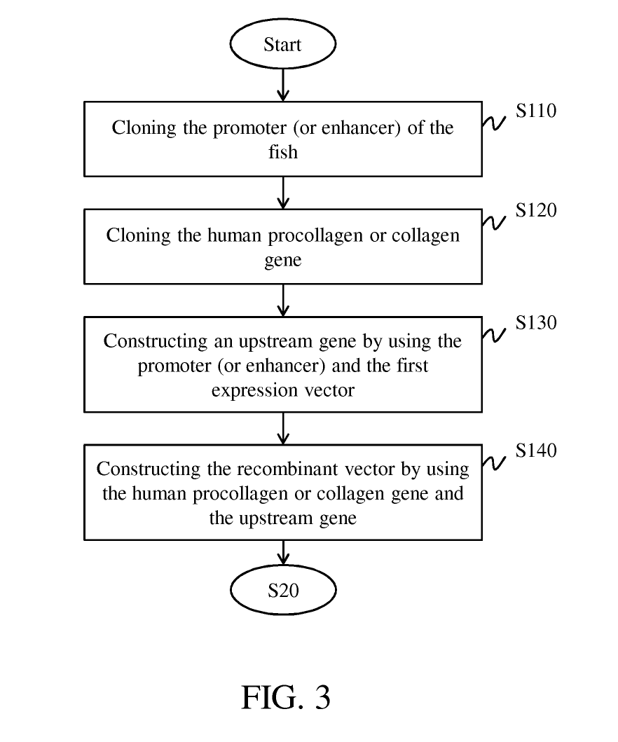

Please refer to FIG. 3, in which in some embodiments, the step S10 includes cloning the promoter (or/and the enhancer) of the fish (step S110), cloning the human procollagen or collagen gene hCG (step S120), constructing the upstream gene UG by using the promoter (or/and the enhancer) and the first expression vector EV (step S130) and constructing the recombinant vector having the human procollagen or collagen gene hCG and the upstream gene UG (step S140).

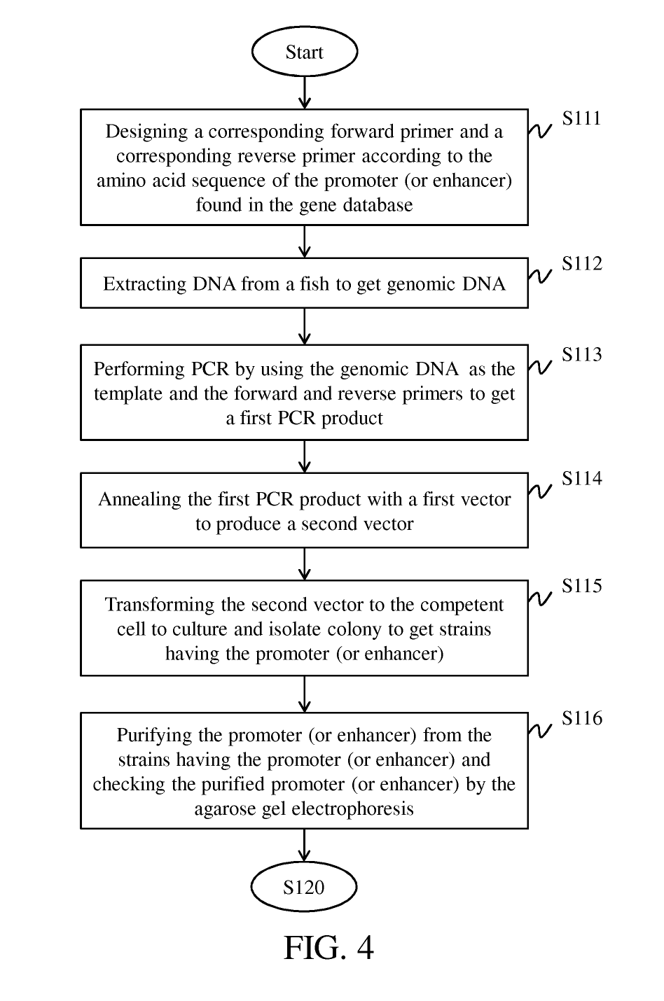

Please refer to FIG. 4, in which in some embodiments, the step S110 includes designing a corresponding forward primer (5′ primer), and a corresponding reverse primer (3′ primer), according to the amino acid sequence of the promoter (or the enhancer) found in the gene database, extracting DNA (deoxyribonucleic acid), from a fish (fish tissue), to obtain genomic DNA (step S112), performing the polymerase chain reaction (PCR) by using the genomic DNA got in step S112 as the template and the forward and reverse primers got in step S111 to react with the reagents, such as the DNA polymerase, deoxyribonucleoside triphsphate (dNTP), and etc., to got a first PCR product (step S113), annealing the first PCR product with a first vector to produce a second vector (step S114), transforming the second vector to the competent cell to culture and isolate colony to obtain strains having the promoter (or the enhancer) (step S115), and purifying the promoter (or the enhancer) from the strains having the promoter (or the enhancer) and checking the purified promoter (or the enhancer) by the agarose gel electrophoresis (step S116).

In some embodiments, the fish can be a Zebra fish, etc. As for the sequence of the SEQ ID NO: 1, for example, the forward primer and the reverse primer can be the sequences shown as SEQ ID NO: 6 and SEQ ID NO: 7, respectively.

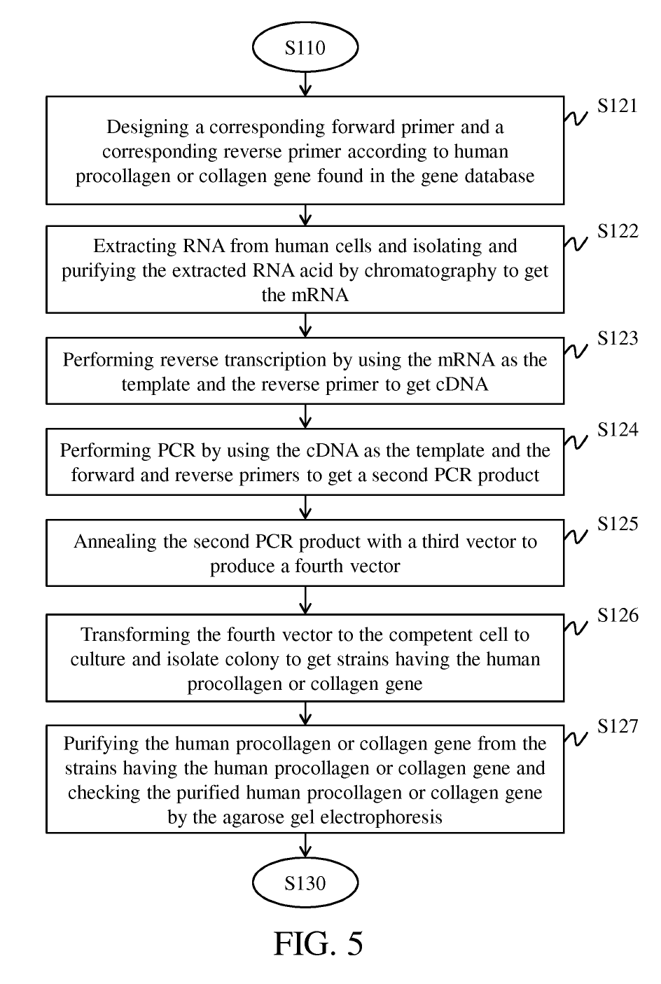

Please refer to FIG. 5, in which in some embodiments, the step S120 includes designing a corresponding forward primer and a corresponding reverse primer according to the message RNA (mRNA) of the human procollagen or collagen gene hCG found in the gene database (step S121), extracting ribonucleic acid (RNA) from human cells and isolating and purifying the extracted RNA by chromatography to obtain the mRNA (step S122), performing reverse transcription (RT) by using the message RNA got in step S122 as the template and the reverse primer get in step S121 as the primer to obtain complementary DNA (cDNA) (step S123), performing the PCR by using the cDNA got in step S123 as the template and the forward and the reverse primers get in step S121 as the primers to react with the reagents, such as the DNA polymerase, deoxyribonucleoside triphsphate and etc., to obtain a second PCR product (step S124), annealing the second PCR product with a third vector to produce a fourth vector (step S125), transforming the fourth vector to the competent cell to culture and isolate colony to obtain strains having the human procollagen or collagen gene hCG (step S126), and purifying the human procollagen or collagen gene hCG from the strains having the human procollagen or collagen gene hCG and checking the purified human procollagen or collagen gene hCG by the agarose gel electrophoresis (step S127).

In some embodiments, the human cell can be the firoblast, the chondrocyte, etc. As for the sequence of the SEQ ID NO: 3, for example, the forward primer can be the sequences shown as SEQ ID NO: 8 and SEQ ID NO: 10, and the reverse primer can be the sequences shown as SEQ ID NO: 9 and SEQ ID NO: 11. As for the sequence of the SEQ ID NO: 4, for example, the forward primer and the reverse primer can be the sequences shown as SEQ ID NO: 12 and SEQ ID NO: 13, respectively. As for the sequence of the SEQ ID NO: 5, for example, the forward primer can be the sequences shown as SEQ ID NO: 14 and SEQ ID NO: 16 and the reverse primer can be the sequences shown as SEQ ID NO: 15 and SEQ IN NO: 17.

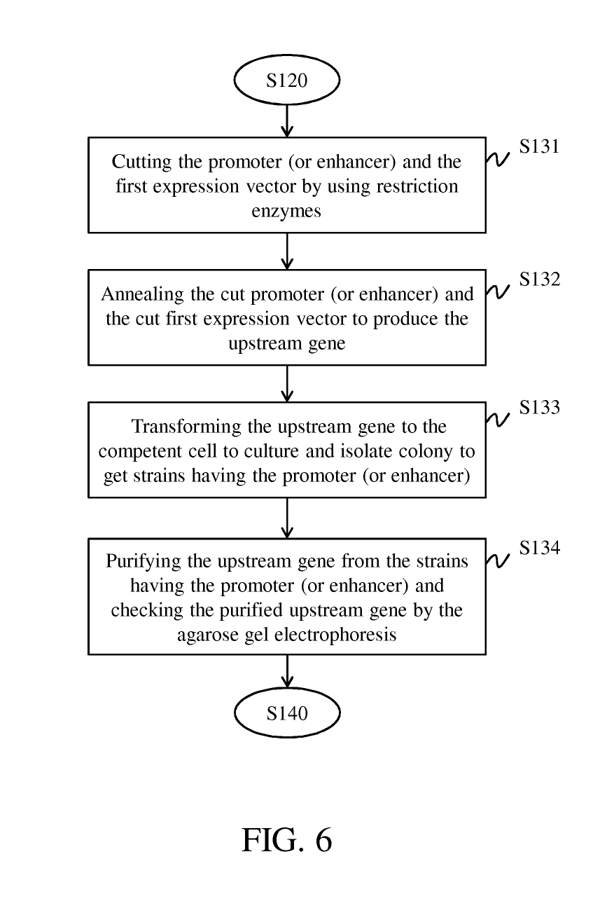

Please refer to FIG. 6, in which in some embodiments, the step S130 includes individually cutting the promoter (or the enhancer) got in step S116 and the first expression vector by using one or two restriction enzymes (step S131), annealing the cut promoter (or the enhancer) and the cut first expression vector EV to produce the upstream gene UG (step S132), transforming the upstream gene UG to the competent cell to culture and isolate colony to obtain strains having promoter (or the enhancer) (step S133), and purifying the upstream gene UG from the strains having promoter (or the enhancer) and checking the purified upstream gene UG by the agarose gel electrophoresis (step S134).

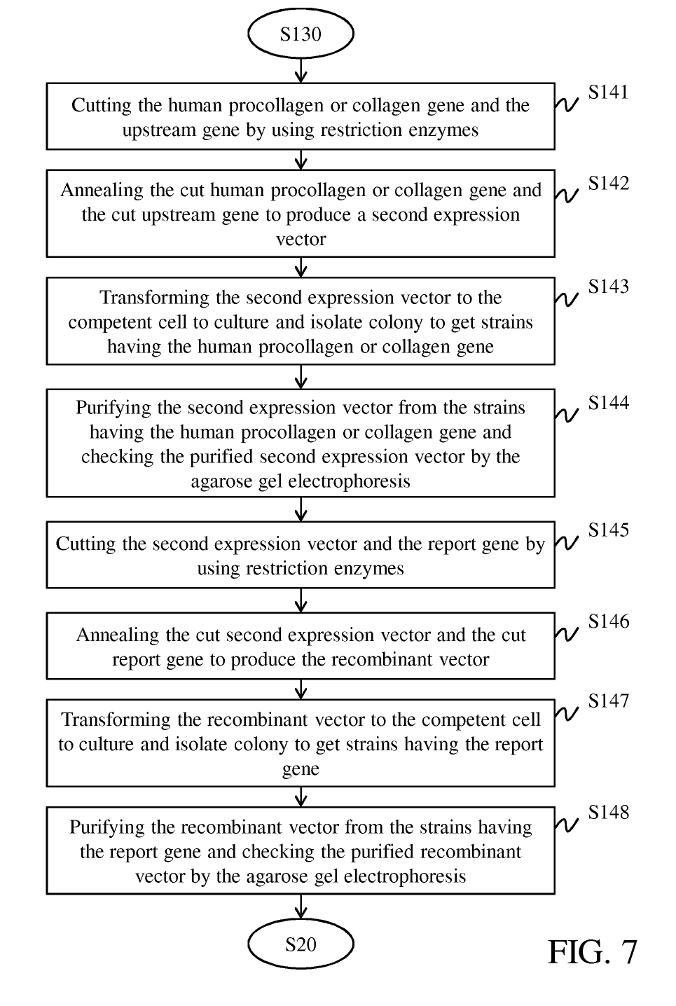

Please refer to FIG. 7, in which in some embodiments, the step S140 includes individually cutting the human procollagen or collagen gene hCG got in step S127 and the upstream gene UG got in step S134 by using one or two restriction enzymes (step S141), annealing the cut human procollagen or collagen gene hCG and the cut upstream gene UG to produce a second expression vector (step S142), transforming the second expression vector to the competent cell to culture and isolate colony to obtain strains having the human procollagen or collagen gene hCG (step S143), purifying the second expression vector from the strains having the human procollagen or collagen gene hCG and checking the purified second expression vector by the agarose gel electrophoresis (step S144), cutting the second expression vector got in step S144 and the report gene RG by using one or two restriction enzymes (step S145), annealing the cut second expression vector and the cut report gene RG to produce the recombinant vector (step S146), transforming the recombinant vector to the competent cell to culture and isolate colony selection to obtain strains having the report gene RG (step S147), and purifying the recombinant vector from the strains having the report gene and checking the purified recombinant vector by the agarose gel electrophoresis (step S148).

In some embodiments, when it is designed that the recombinant vector does not have the report gene RG, the steps from S145 to S148 can be omitted, and at this time, the second expression vector got in step S144 is the recombinant vector.

Here, as the proper competent cells are known by those skilled in the arts, and many competent cells are commercially available, such as prokaryotic cell, eucaryotic cell or etc, therefore, further details about the competent cells are not provided.

As should known by those skilled in the arts, the transgenic fish egg is the embryo in which the aforementioned recombinant vector is artificially introduced, or is a fish egg spawned by a fish grown from the transgenic embryo. For convenience, the embryo in which the aforementioned recombinant vector is artificially introduced and the coming generations thereof are generally called as the transgenic fish hereafter. That is, the cell of the transgenic fish has at least one clone body of the recombinant vector. For convenience, the recombinant vector artificially introduced and the clone bodies thereof are generally called as the recombinant gene structure hereafter.

Consequently, according to the types of the upstream gene UG selected, the upstream gene UG in the clone body can directly or indirectly drive the human procollagen or collagen gene hCG in the clone body to secret the recombinant human procollagen or collagen in the fish scales, the fish skins, the fish eyes or other part(s) of the transgenic fish. Further, the part(s) of the transgenic fish where the recombinant human procollagen or collagen is secreted can treated as the biomaterials (e.g. the biomaterial having the secreted recombinant human procollagens or collagens and inorganic substances) and/or can be treated to purify the recombinant human procollagen or collagen.

Here, each secreted recombinant human procollagen can be one of type I-XXVIII procollagens. Additionally, each secreted recombinant human collagen can be one of the type I-XXVIII collagens or the combination formed with at least two of the type I-XXVIII collagens. Further, all the three chains of the secreted recombinant human collagen can be oriented from the human procollagen or collagen gene hCG. Alternatively one or two of the three chains of the secreted recombinant human procollagen or collagen are oriented from the human procollagen or collagen gene hCG and rest chain(s) thereof are oriented from fish procollagen or collagen gene.

In some embodiments, the part(s) of the transgenic fish having the recombinant human procollagens or collagens can be fish scales, fish skins, fish eyes or the combination thereof.

Hereafter, some practical experiments are described as detailed explanations about the disclosure, but not limited thereto. In experiments, the DNA sequencing is done by Tri-I Biotech, Inc., and the used DNA analyzers system is ABI 3730 Genetic Analyzer. The LB (Luria-Bertaini) medium is prepared by dissolving 20 g LB powder (Bio Basic Inc.) in dH2O, and then adding dH2O to total volume of 1 L. The prepared LB medium is sterilized in autoclave. The LB powder includes 10 g Trypton, 5 g Yeast extract and 5 g NaCl (Sodium chloride). LB/Amp (LB plate with ampicillin) medium is prepared by adding 50 μg/mL ampicillin into LB medium.

First Experiment: Extracting DNA from a Fish

The genomic DNA is extracted from the wild-type zebrafish (Danio rerio AB strain) by a GenoMaker kit. The GenoMaker kit includes GenoMaker reagent, RNase cocktail and Ethanol precipitate Buffer.

Firstly, the zebrafish tissue is ground by the liquid nitrogen grinding technique or by the homogenizer. Next, 1 ml GenoMaker reagent is added to about 10 mg to 100 mg ground fish tissue to lysis the cells, to obtain the lysis homogeneous sample. Then, the lysis homogeneous sample (1 ml) is incubated in room temperature for 3 to 5 minutes and 500 μl phenol/chloroform (v:v=1:1) is added to and mixed uniformly with the lysis homogenous sample to obtain a first mixed solution. The first mixed solution is centrifuged at 12000 rpm for 5 minutes, and the supernatant liquid of the first mixed solution is carefully absorbed and transferred to a new centrifuge tube. Further, 0.7 to 0.8 ml isopropanol is added to the centrifuge tube and uniformly mixed with the supernatant liquid of the first mixed solution to obtain a second mixed solution. The second mixed solution is centrifuged at 12000 rpm for 5 minutes, and the supernatant liquid of the second mixed solution is removed and the precipitates are left in centrifuge tube. 40 μl RNase cocktail is added to the centrifuge tube having precipitates and incubated in 37° C. for 5 minutes to dissolve the subhumid precipitates. Next, 600 μl Ethanol precipitate Buffer is added to the centrifuge tube and uniformly mixed with the precipitate solution to obtain a third mixed solution. The third mixed solution is centrifuged at 12000 rpm for 5 minutes, and the supernatant liquid is removed and the precipitates are left in the centrifuge tube. Finally, 50 to 200 μl TE buffer is added to the centrifuge tube to dissolve the precipitates and then the genomic DNA of the fish is acquired. The genomic DNA is stored in −20° C. for further experiments.

Second Experiment: Cloning the Promoter of the Fish

Polymerase Chain Reaction:

K4 promoter shown as SEQ ID NO: 1 is taken as an example. Here, the sequence of SEQ ID NO: 6 is applied as the forward primer, and the sequence of SEQ ID NO: 7 is applied as the reverse primer.

According to Table 1, reagents are added into a 0.2 ml micro test tube to perform PCR to obtain the first PCR product. Here, the first stage condition of PCR is reacting at 94° C. for 1 minute; the second stage condition is reacting at 58° C. for 1 minute; the third stage condition is reacting at 72° C. for 10 seconds, and each stage is progressed in order for 35 cycles.

Then, the first PCR product is checked by 1% agarose gel electrophoresis to ensure the K4 promoter is present in the first PCR product or not.

TABLE 1

| Reagent | Usage amount | |
|---|---|---|
| Template DNA (10-times diluted Genomic DNA of | 2 | μl |
| the fish) | | |
| 5′ primer (3 pmol/μl) | 3 | μl |
| 3′ primer (3 pmol/μl) | 3 | μl |
| 10X Taq DNA polymerase buffer | 5 | μl |
| Taq DNA polymerase (SuperTag ™XL) | 0.5 | μl |
| 2.5 mM dNTP | 4 | μl |
| Sterile H₂O | 32.5 | μl |
| Total amount | 50 | μl |

Annealing Reaction:

According to Table 2, reagents are added into a 0.2 ml micro test tube to perform the annealing reaction (4° C., overnight), to obtain the fourth mixed solution (which contains the second vector).

TABLE 2

| Reagent | Usage amount |
|---|---|
| First PCR product | 4 μL |
| yT&A vector (first vector) | 1 μL |
| T4 DNA ligase buffer (A) | 1 μL |
| T4 DNA ligase buffer (B) | 1 μL |
| T4 DNA ligase | 1 μL |
| Sterile H₂O | 2 μL |
| Total amount | 10 μL |

Transformation Reaction:

Here, the used competent cells are the ECOS 101 (E. coli) strains.

10 μl fourth mixed solution is added into and mixed uniformly with 100 μl competent cell to produce a first bacterial solution. After mixing, immediately put the first bacterial solution into an ice bath for about 5 minutes, and followed by putting the first bacterial solution into 42° C. for 45 seconds to proceed the heat-shock reaction. After the heat-shock reaction, the first bacterial solution is put into the ice bath again for 5 minutes. Lastly, the ice bathed first bacterial solution is uniformly coated in the LB/Amp medium, to incubate at 37° C. overnight.

Colony Selection:

The colony grown in the aforementioned LB/Amp medium is re-cultured in a new LB/Amp medium and incubated at 37° C. overnight.

First Plasmid Purification:

Here, the first plasmid is prepared by the Plasmid Mini-prep Purification kit (bought from Protech Technology Enterprise Co., Ltd.).

Firstly, 1.5 ml second bacterial solution is taken out to centrifuge at 12000×g for 5 minutes. After the centrifugation, the bacterial precipitates are taken out.

200 μl solution I (resuspension solution), is added into the micro centrifuge tube contained with the bacterial precipitates, and then the micro centrifuge tube is oscillated until the bacteria are suspended. And 200 μl solution II (lysis solution), is added into the micro centrifuge tube and mixed with the bacterial solution contained in the micro centrifuge tube to dissolve the bacteria completely to isolate the first plasmid. Then, 200 μl solution III (neutralization solution), is added into the micro centrifuge tube and mixed with the bacterial solution contained in the micro centrifuge tube. After the addition, the bacterial solution is centrifuged at 12000×g for 5 minutes and the supernatant liquid of the bacterial solution is taken out and added to a spin column (which has DNA absorbing film), and a collection tube. Then, the spin column and the collection tube having the supernatant liquid of the bacterial solution is centrifuged at 12000×g for 1 minute, so that the DNA in the supernatant liquid can be absorbed on the DNA absorbing film. Subsequently, 350 ml wash solution is added to the spin column and the spin column is centrifuged at 12000×g for 1 minute, the aforementioned step is repeated again. Then, the spin column is centrifuged at 12000×g for 3 minutes to remove the residual ethanol. Subsequently, the spin column is put into a new 1.5 ml micro centrifuge tube, and 50 μl elution buffer which is preheated to 68° C. is added to the micro centrifuge tube and incubated for 5 minutes. Then, the micro centrifuge tube is centrifuged at 12000×g for 3 minutes, and the DNA (namely, the first plasmid), in the precipitates after the centrifugation is collected; the size of the first plasmid is checked by the restriction enzyme. 10-times diluted reaction buffer and water are added into appropriate amounts of DNA and restriction enzyme (BamH I and Sal I), to form a fifth mixed solution with the volume being 15 μl. The fifth mixed solution is reacted at 37° C. for at least one hour, and then added into 1% agarose gel for the electrophoresis analysis, to ensure the DNA segments of the K4 promoter to be embedded is present in the first plasmid.

Third Experiment: Cloning the Human Type II Procollagen or Collagen Gene

Polymerase Chain Reaction:

The human type II procollagen or collagen gene (hCollagen type 2 alpha 1, hCT2A1) shown as SEQ ID NO: 5 is taken as an example. Here, the sequence of SEQ ID NO: 14 is applied as the forward primer, and the sequence of SEQ ID NO: 15 is applied as the reverse primer.

Reagents according to Table 3 are added into a 0.2 ml micro test tube to proceed polymerase chain reaction to obtain the second PCR product. Here, the first stage condition of PCR is reacting at 98° C. for 10 seconds; the second stage condition is reacting at 64° C. for 30 seconds; the third stage condition is reacting at 72° C. for 4 minutes and 30 seconds, and each stage is progressed in order for 30 cycles.

Then, the second PCR product is checked by 1% agarose gel electrophoresis, to ensure the human procollagen or collagen gene is present in the second PCR product or not.

TABLE 3

| Reagent | Usage amount | |
|---|---|---|
| Template DNA (16 ng/μl) | 10 | μL |
| 5′ primer (2 μM) | 5 | μL |
| 3′ primer (2 μM) | 5 | μL |
| TAQ + polymerase buffer + dNTP Mixture | 25 | μL |
| Sterile H₂O | 5 | μL |
| Total amount | 50 | μL |

Wherein, the template DNA is the cDNA of the human procollagen or collagen gene.

Annealing Reaction:

According to Table 4, reagents are added into a 0.2 ml micro test tube to proceed annealing reaction (22° C., 4 hours), to obtain the sixth mixed solution (which contains the fourth vector).

TABLE 4

| Reagent | Usage amount | |
|---|---|---|
| Second PCR product | 6.5 | μL |
| yT&A vector (third vector) | 1 | μL |
| T4 DNA ligase buffer (A) | 1 | μL |
| T4 DNA ligase buffer (B) | 1 | μL |
| T4 DNA ligase | 0.5 | μL |
| Total amount | 10 | μL |

Transformation Reaction:

The sixth mixed solution is added to the prepared E. coli TOP 10 bacterial solution, and then the mixture is put on the ice for 30 minutes. Then, the mixture is water bathed at 42° C. for 1-2 minutes to proceed the heat-shock reaction. After the heat-shock reaction, the mixture is ice bathed immediately for 3-5 minutes, and then LB medium is added to the mixture to total volume of 1 L, to obtain a third bacterial solution. The third bacterial solution is undergone agitation culture at 37° C. for 1 hour. 100 μl of 100 mM IPTG (isopropyl-b-d-thiogalactopyranoside) and 20 μl of 50 mg/ml X-gel (5-bromo-4-chloro-3-indolyl-beta-D-galactopyranoside) are coated on the LB/Amp medium, and the third bacterial solution is uniformly coated in the LB/Amp medium to incubate at 37° C. for 12-16 hours.

Here, the quick screening, such as blue white screen, etc., can be applied to roughly isolate the strains having the human procollagen or collagen gene. As the procedure of the quick screening is well known by those skilled in the art, additional details relating to the procedure of the quick screening are not provided.

### Second Plasmid Purification

A white single colony is picked up by micropipettes and is mixed with 10 ml LB/amp medium in the test tube to incubate for 8 hours. Then, several colonies are chosen and undergone agitation culture at 37° C. for 14 hours, to be prepared for purifying the second plasmid having the DNA segments of the human type II procollagen or collagen gene (referred to hCT2A1-yt&A). Here, the second plasmid is prepared by the Plasmid Mini-prep Purification kit (bought from Protech Technology Enterprise Co., Ltd.), and the size of the second plasmid is checked by the restriction enzyme (BamH and Sal I). Subsequently, 1% agarose gel is applied in the electrophoresis analysis, to ensure the DNA segments of the human type II procollagen or collagen gene to be embedded is present in the second plasmid. As the purification procedure is well known by those skilled in the art, additional details relating to the purification procedure are not provided.

Fourth Embodiment: Constructing the Upstream Gene Applicable for the Human Type II Procollagen or Collagen Gene

According to Table 5, restriction enzymes (ApaI and BamH I), is applied to cut the first plasmid (K4-yt&A) and the first expression vector (pT2-LFABP-tTa) (reacting at 37° C. for at least 2 hours), to obtain a seventh mixed solution (which contains K4), and an eighth mixed solution (which contains pT2-tTa), respectively.

TABLE 5

| Reagent | Usage amount | |
|---|---|---|
| K4-yt&A or pT2-LF-tTa | 16 | μl |
| Apa1 | 1 | μl |
| BamH 1 | 1 | μl |
| T4 DNA ligase Buffer A | 2 | μl |
| Total amount | 20 | μl |

After the reaction, the seventh mixed solution (which contains K4), and the eighth mixed solution (which contains pT2-tTa), are injected into 1% agarose gel to undergo electrophoresis analysis respectively, and DNA segments with certain sizes are cut from the seventh mixed solution and the eighth mixed solution, respectively. Then, DNA products (that is, K4 and pT2-tTa), are recycled by the Gel Elution Kit (bought from GeneMark).

According to Table 6, the two DNA products (K4 and pT2-tTa), are mixed at 70° C. for 3 minutes, and then the mixture is ice bathed immediately. Subsequently, other reagents are added into the mixture uniformly; and the mixture is incubated at 22° C. for 4 hours to undergo the annealing reaction, to obtain a ninth mixed solution (which contains the upstream gene).

TABLE 6

| Reagent | Usage amount | |
|---|---|---|
| K4 | 7.5 | μl |
| pT2-tTa | 1.5 | μl |
| 2 ligase buffer | 10 | μl |
| T4 DNA ligase | 1 | μl |
| Total amount | 20 | μl |

The E. coli TOP 10 strains are applied as the competent cells, and the upstream gene in the ninth mixed solution is transformed to the E. coli TOP 10 to incubate (strain culture). Subsequently, strains having the upstream gene are selected from the cultivated strains and purified by the purified kit to obtain the upstream gene (pT2-K4-tTa).

As the procedures of the strain culture, the strain isolation and the purification are well known by those skilled in the art, additional details relating to these procedures are not provided.

Fifth Experiment: Constructing the Recombinant Plasmid

According to Table 7, restriction enzymes (Sal I and BamH I), are applied to cut pT2-K4-tTa and hCT2A1-yt&A (at 37° C. for at least two hours), respectively to obtain a tenth mixed solution (which contains pT2-K4), and an eleventh mixed solution (which contains hCT2A1).

TABLE 7

| Reagent | Usage amount | |
|---|---|---|
| pT2-K4-tTa or hCT2A1-yt&A | 16 | μl |
| Sal I | 1 | μl |
| BamH I | 1 | μl |
| Restriction Enzyme Buffer | 2 | μl |
| Total amount | 20 | μl |

After the reaction, the tenth mixed solution (which contains pT2-K4), and the eleventh mixed solution (which contains hCT2A1), are injected into 1% agarose gel to undergo electrophoresis analysis respectively, and DNA segments with certain sizes are cut from the tenth mixed solution and the eleventh mixed solution, respectively. Then, DNA products (that is, pT2-K4 and hCT2A1), are recycled by the Gel Elution Kit (DNA recycle kit, bought from GeneMark).

According to Table 8, the two DNA products (pT2-K4 and hCT2A1), are mixed at 70° C. for 3 minutes, and then the mixture is ice bathed immediately. Subsequently, other reagents are added into the mixture uniformly; and the mixture is incubated at 22° C. for 4 hours to undergo the annealing reaction to obtain a twelfth mixed solution (which contains the second expression vector).

TABLE 8

| Reagent | Usage amount | |
|---|---|---|
| hCT2A1 | 7.5 | μl |
| pT2-K4 | 1.5 | μl |
| 2 ligase buffer | 10 | μl |
| T4 DNA ligase | 1 | μl |
| Total amount | 20 | μl |

The E. coli TOP 10 strains are applied as the competent cells, and the second expression vector in the twelfth mixed solution is transformed to the E. coli TOP 10 to incubate (strain culture). Subsequently, strains having the second expression vector are selected from the cultivated strains and purified by the purified kit to obtain the second expression vector (pT2-K4-hCT2A1).

Here, the green fluorescent protein gene is used as the report gene.

According to Table 9, restriction enzymes (Sal I and Cla I), are applied to cut the report gene (IRES-hrGFP), and pT2-K4-hCT2A1 (at 37° C. for at least two hours), respectively to obtain a thirteenth mixed solution (which contains IRES-hrGFP), and a fourteenth mixed solution (which contains pT2-K4-hCT2A1).

TABLE 9

| Reagent | Usage amount | |
|---|---|---|
| IRES-hrGFP or pT2-K4-hCT2A1 | 16 | μl |
| Sal I | 1 | μl |
| Cla I | 1 | μl |
| Restriction Enzyme Buffer | 2 | μl |
| Total amount | 20 | μl |

After the reaction, the thirteenth mixed solution (which contains IRES-hrGFP), and the fourteenth mixed solution (which contains pT2-K4-hCT2A1), are injected into 1% agarose gel to undergo electrophoresis analysis respectively, and DNA segments with certain sizes are cut from the thirteenth mixed solution and the fourteenth mixed solution, respectively. Then, DNA products (that is, IRES-hRGEP and pT2-K4-hCT2A1), are recycled by the Gel Elution Kit (DNA recycle kit, bought from GeneMark).

According to Table 10, the two DNA products (IRES-hRGEP and pT2-K4-hCT2A1), are mixed at 70° C. for 3 minutes, and then the mixture is ice bathed immediately. Subsequently, other reagents are added into the mixture uniformly; and the mixture is incubated at 22° C. for 4 hours to undergo the annealing reaction to obtain a fifteenth mixed solution (which contains the recombinant plasmid).

TABLE 10

| Reagent | Usage amount | |
|---|---|---|
| IRES-hrGFP | 7.5 | μl |
| pT2-K4-hCT2A1 | 1.5 | μl |
| 2 ligase buffer | 10 | μl |
| T4 DNA ligase | 1 | μl |
| Total amount | 20 | μl |

The E. coli TOP 10 strains are applied as the competent cells, and the recombinant plasmid in the fifteenth mixed solution is transformed to the E. coli TOP 10 to incubate (strain culture). Subsequently, strains having the recombinant plasmid are selected from the cultivated strains. As the procedures of the strain culture and the strain isolation are well known by those skilled in the art, additional details relating to these procedures are not provided.

Herein, after the recombinant plasmid is transformed to the E. coli TOP 10, the cryopreservation medium (20% Glycerol: LB medium=1:1) can be added.

Recombinant Plasmid Purification:

The strains which are selected and sequencing ensured are inoculated into 200 ml LB/Amp medium to undergo agitation culture at 37° C. with 175 rpm. After the agitation culture, the mixture is centrifuged at 13000×g for 5 minutes to obtain the bacterial precipitates.

10 ml equilibration buffer is added into the balance column in advance. 4 ml cell suspension buffer is added into the centrifuged bacterial precipitates to oscillate until no precipitates exists (that is, all the bacterial precipitates is dissolved), to obtain a sixteenth mixed solution.

4 ml cell lysis solution is added and carefully mixed with the sixteenth mixed solution; then the mixture is incubated in room temperature for 5 minutes. Next, 4 ml neutralization buffer solution is added and carefully mixed with the mixture and carefully mixed with the mixture, and then the mixture is centrifuged at 15000×g for 10 minutes (25° C.). After the centrifugation, the supernatant liquid of the mixture is absorbed and transferred to the aforementioned balance column and washed by using 10 ml wash solution for two times, and then 5 ml elution buffer is added to the balance column and the DNA solution eluted out is collected.

Subsequently, 3.5 ml isopropanol is added to the collected DNA solution and the mixture is centrifuged at 15000×g for 30 minutes. After the centrifugation, the supernatant liquid is poured out and 3 ml 70% alcohol is added to the precipitates left. Then, the mixture is centrifuged at 15000×g for 5 minutes so as to remove the alcohol. After the alcohol is removed, 200 μl TE buffer is added to the air-dried DNA to dissolve the DNA, then the solution which contains the recombinant plasmid (pT2-K4-hCT2A1-IRES-hrFGP) is obtained. After the DNA is dissolved completely, 2 μl solution is taken out to measure the optical density at 260 nm (OD 260 nm), so as to quantify the DNA, and the rest solution is stored at −80° C.

The cooling procedure includes sequentially storing it at 4° C. for 1 hour, at −20° C. for 1 hour, and at −80° C. for 1 hour.

Sixth Experiment: Cloning the Human Type I Procollagen or Collagen Gene

The human type I procollagen or collagen gene (hCollagen type 1 alpha 1, hCT1A1), shown as SEQ ID NO: 3 is taken as an example. Here, the sequence of SEQ ID NO: 8 is applied as the forward primer, and the sequence of SEQ ID NO: 9 is applied as the reverse primer.

As the procedures of cloning the human type I procollagen or collagen gene is substantially similar to the procedures of cloning the human type II procollagen or collagen gene as described in the third experiment, additional details relating to these procedures are not provided. In this experiment, the second plasmid having the DNA segments of the human type I procollagen or collagen gene is obtained, referred to as hCT1A1-yt&A.

Seventh Experiment: Constructing the Upstream Gene Applicable for the Human Type I Procollagen or Collagen Gene

According to Table 11, restriction enzymes (Apa I and BamH I), are applied to cut the first plasmid (K4-yt&A), and the first expression vector (pT2-LF2.9-AmCyan1) (at 37° C. for at least two hours), respectively to obtain a seventeenth mixed solution (which contains K4), and an eighteenth mixed solution (which contains pT2-AmCyan1).

TABLE 11

| Reagent | Usage amount | |
|---|---|---|
| K4-yt&A or pT2-LF2.9-AmCyan1 | 16 | μl |
| Apa I | 1 | μl |
| BamH I | 1 | μl |
| Buffer A | 2 | μl |
| Total amount | 20 | μl |

After the reaction, the seventeenth mixed solution (which contains K4), and the eighteenth mixed solution (which contains pT2-AmCyan1), are injected into 1% agarose gel to undergo electrophoresis analysis respectively, and DNA segments with certain sizes are cut from the seventeenth mixed solution and the eighteenth mixed solution, respectively. Then, DNA products (that is, K4 and pT2-AmCyan1), are recycled by the Gel Elution Kit (DNA recycle kit, bought from GeneMark).

According to Table 12, the two DNA products (K4 and pT2-AmCyan1) are mixed at 70° C. for 3 minutes, and then the mixture is ice bathed immediately. Subsequently, other reagents are added into the mixture uniformly; and the mixture is incubated at 22° C. for 4 hours to undergo the annealing reaction to obtain a nineteenth mixed solution (which contains the second expression vector).

TABLE 12

| Reagent | Usage amount | |
|---|---|---|
| K4 | 7.5 | μl |
| pT2-AmCyan1 | 1.5 | μl |
| 2 ligase buffer | 10 | μl |
| T4 DNA ligase | 1 | μl |
| Total amount | 20 | μl |

The E. coli TOP 10 strains are applied as the competent cells, and the second expression vector in the nineteenth mixed solution is transformed to the E. coli TOP 10 to incubate (strain culture). Subsequently, strains having the upstream gene are selected from the cultivated strains and purified by the purified kit to obtain the upstream gene (pT2-K4-AmCyan1).

As the procedures of the strain culture, the strain isolation and the purification are well known by those skilled in the art, additional details relating to these procedures are not provided.

Eighth Experiment: Constructing the Recombinant Plasmid

According to Table 13, restriction enzymes (Sal I and Age I), are applied to cut pT2-K4-AmCyan1 and hCT1A1-yt&A (at 37° C. for at least two hours), respectively to obtain a twentieth mixed solution (which contains pT2-K4), and a twenty-first mixed solution (which contains hCT1A1).

TABLE 13

| Reagent | Usage amount | |
|---|---|---|
| pT2-K4-AmCyan1 or hCT1A1-yt&A | 16 | μl |
| Sal I | 1 | μl |
| Age I | 1 | μl |
| Buffer D | 2 | μl |
| Total amount | 20 | μl |

After the reaction, the twentieth mixed solution (which contains pT2-K4), and the twenty-first mixed solution (which contains hCT1A1), are injected into 1% agarose gel to undergo electrophoresis analysis respectively, and DNA segments with certain sizes are cut from the twentieth mixed solution and the twenty-first mixed solution, respectively. Then, DNA products (that is, pT2-K4 and hCT1A1), are recycled by the Gel Elution Kit (DNA recycle kit, bought from GeneMark).

According to Table 14, the two DNA products (that is, pT2-K4 and hCT1A1), are mixed at 70° C. for 3 minutes, and then the mixture is ice bathed immediately. Subsequently, other reagents are added into the mixture uniformly; and the mixture is incubated at 22° C. for 4 hours to undergo the annealing reaction to obtain a twenty-second mixed solution (which contains the recombinant plasmid).

TABLE 14

| Reagent | Usage amount | |
|---|---|---|
| hCT1A1 | 7.5 | μl |
| pT2-K4 | 1.5 | μl |
| 2 ligase buffer | 10 | μl |
| T4 DNA ligase | 1 | μl |
| Total amount | 20 | μl |

The recombinant plasmid in the twenty-second mixed solution is transformed to the E. coli TOP 10 to incubate (strain culture). Subsequently, strains having the recombinant plasmid are selected from the cultivated strains and purified by the purified kit to obtain the recombinant plasmid (referred to as pT2-K4-hCT1A1).

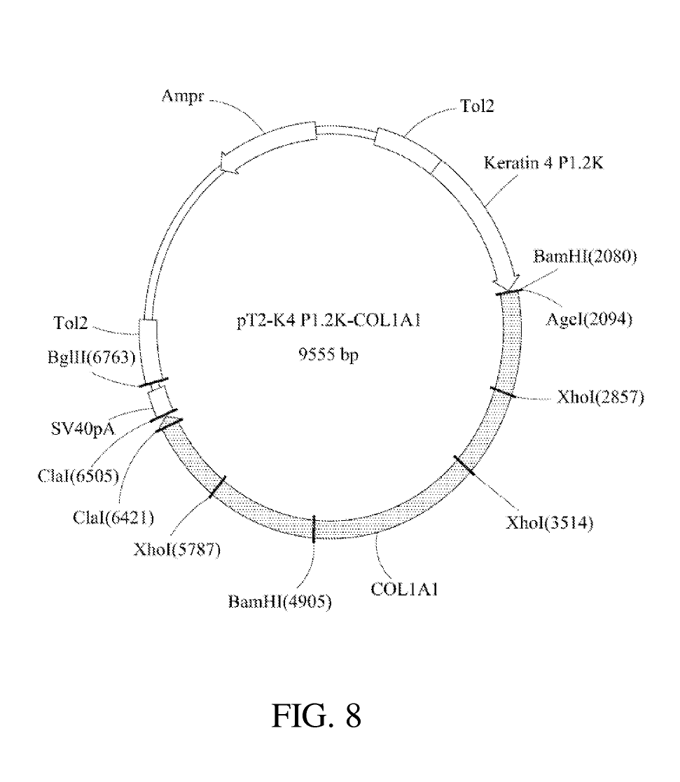

Then, the recombinant plasmid of pT2-K4-hCT1A1 is transformed to the E. coli TOP 10, to incubate (strain culture). Subsequently, strains having the recombinant plasmid of pT2-K4-hCT1A1, as shown in FIG. 8, are selected from the cultivated strains and purified by the purified kit. In this experiment, the procedures are substantially similar as that described in the fifth experiment.

In FIG. 8, K4 P1.2K (keratin 4 P1.2K) refers to K4 1.2 kb promoter of the Zebra fish, COL1A1 refers to cDNA of the human type I, alpha 1 collagen, and Tol2 refers to Tol2 transposase recognition sequence for transposition. A cutting site of LF2.9 promoter ApaI/BamHI of pT2-LF2.9-AmCyan1 is displaced by the K4 promoter ApaI/BamHI of the Zebra fish, to form pT2-K4-AmCyan1. A cutting site of Amcyan1 AgeI/XhoI of the pT2-K4-AmCyan1 is displaced by hCT1A1 AgeI/SalI, to obtain the pT2-K4-hCT1A1. Herein, cutting sites of Sail and XhoI are isoschizomers, and both SalI and XhoI are destroyed after ligation.

Ninth Experiment: Preparation of the Fish Embryos:

Wild type grown-up zebrafishes (Danio rerio, AB strain), are raised at 28° C., the condition of the photoperiod is set as 14 hours daylight/10 hours darkroom, with raising feeding stuffs at the morning and the evening.

The male zebrafishes and the female zebrafishes are mixed cultured at the morning regularly (male:female=2:1), to copulate with each other to lay eggs. After the copulation is finished for about 30 to 40 minutes, the embryos are collected (at this time, the embryos are at the 1-cell stage), to be applied in gene transfection.

Tenth Experiment: Preparation of the Recombinant Plasmid to be Introduced:

According to Table 15, reagents are mixed uniformly and incubated at 37° C. to react with each other for at least 3 hours, to convert the constructed recombinant plasmid (as the product of the fifth embodiment or the eighth embodiment) into linear DNA by using proper restriction enzymes.

TABLE 15

| Reagent | Usage amount |
|---|---|
| Recombinant plasmid | Appropriate |
| | amount |
| Restriction enzyme | 2 μl |
| BSA (10 mg/ml) | 1 μl |

Sterile water is added until total volume is 20 μl

Gel Elution Kit (bought from GeneMark), is applied to recycle the DNA products, and appropriate amount of sterile water is added to re-dissolve DNA to make the final concentration of the linear DNA is 0.1 μg/μl.

Before the microinjection, 40 μl linear DNA is uniformly mixed with 5 μl tracing dye (here, phenol red is applied as the tracing dye), to obtain a twenty-third mixed solution. The twenty-third mixed solution is stored at −20° C. for the microinjection.

Eleventh Experiment: Introducing the Recombinant Plasmid

The collected embryos are disposed in the groove of the agarose gel, so that the embryos are incubated on the agarose gel. Then, the aforementioned twenty-third mixed solution is injected into the incubated embryos via microinjection.

After the microinjection, the embryos are moved to the incubation tank and cultivated for 24 hours. Then, the green fluorescent protein (hrGFP), expressed on the embryos are observed by the fluorescent microscope and are photographed for record. Here, the expression of the green fluorescent protein means the expression of the human procollagen or collagen gene.

The transgenic embryos are grown as the parent-generation (F0 generation) transgenic zebrafishes. Then, the F0 generation transgenic zebrafishes are grown to be sexually matured (about 3 to 5 months), and copulated with wild type stock zebrafishes to get the F1 generation (child generation), transgenic zebrafishes which express fluorescence. Then, after the F1 generation transgenic zebrafishes are raised for 3 to 5 months, the F1 generation transgenic zebrafishes can copulate to produce the F2 generation (child generation) transgenic zebrafishes.

Here, K4 can drive the human procollagen or collagen gene to express on the fish scales; that is, to make the transgenic zebrafishes secret the human procollagen or collagen gene on the fish scales.

Here, the report gene is applied to ensure the expression of the introduced recombinant gene structure, but embodiments of the disclosure are not limited thereto. For those who are skilled in the art knows that the fishes having the recombinant gene structures can be checked by many proper methods. For example, the chromosomes of the fishes are checked by PCR, to detect whether the fishes have the recombinant gene structures or not. Or, the expression product (the human procollagen or collagen), of the recombinant gene structure can be detected so that the fishes which express the transgenic genes practically can be detected. In addition, related checking method applicable for the transgenic animals can also be applied for the identification of the transgenic fishes, for instance, northern blot or southern blot methods are applied to detect whether the fishes have the nucleic acids of transgenic genes. Or, PCR and other nucleic acid amplification techniques with sequence specificity can be applied.

Twelfth Experiment: Introducing the Recombinant Plasmid

Further, as referring to the gene transfection techniques developed by the Japanese scholar Kawakami, Tol2 transponase mRNA are synthesized by in-vitro transcription and injected into the embryos of the zebra fishes along with the twenty-third mixed solution. The gene transfection techniques can be refer to the publication “Transposon tools and methods in zebrafish Dev. Dyn. 234(2): 224-254, 2005”.

Thirteenth Experiment: Preparation of the Biomaterial

One or many fish scales are collected from the transgenic fish, and the collected fish scales are undergone the washing procedure, the cell removing procedure, the dehydration procedure and other procedures, so that the collected fish scale(s) is/are treated to be a biomaterial having the human procollagen or collagen.

Here, an extrusion procedure can be further undergone to extrude the aforementioned fish scale(s) to be certain shaped and/or certain sized biomaterials having the human procollagens or collagens.

Further, before the extrusion procedure, a grinding procedure can be applied to grind the fish scale(s). In addition, after the grinding procedure, the fish scale particles can be filtered to be separated by different sizes, so that the biomaterials having the human procollagens or collagens can be provided in required sizes.

Furthermore, the aforementioned biomaterials can be sterilized, so that the sterilize biomaterials can be prepared.

The procedures related to the preparation of the aforementioned biomaterials can be referred to U.S. patent application Ser. No. 12/324,551, U.S. patent application Ser. No. 12/659,282, U.S. patent application Ser. No. 12/880,349 and U.S. patent application Ser. No. 13/019,134.

Fourteenth Experiment: Preparation of the E. Coli TOP 10 Strains

Here, the E. coli TOP 10 strains are used as the competent cells, and the genotype thereof is F⁻mcrA Δ(mrr-hsdRMS-mcrBC) φ80lacZΔM15 ΔlacX74 recA1 araD139 Δ(ara-leu) 7697 galU galK rpsL (StrR) endA1 nup^{Gλ-}.

E. coli TOP 10 is cultured in LB medium overnight, and then the bacterial solution is added into 5 ml LB/Amp medium (LB/Amp medium:bacterial solution=100:1) to incubate at 37° C. with vigorous shaking in an incubator until OD600 of 0.5-0.6.

The following steps are operated on ice. The E. coli TOP 10 bacterial solution is stayed on ice for 10 minutes, and the 2 ml of the bacterial solution having O.D600 of 0.5-0.6 is centrifuged at 4000×g, 4° C. for 3 minutes or at 5000×g, 4° C. for 4 minutes. After the centrifugation, the supernatant liquid is removed by a pippet, and then is mix uniformly with 1 ml 200 mM (or 250 mM) CaCl₂, so as to completely re-dissolved the bacteria. The re-dissolved solution is put on the ice for 10 minutes, and then centrifuged at 5000×g, 4° C. for 4 minutes. After the centrifugation, the supernatant liquid is removed and then added with 150 μl-200 ml CaCl₂ to re-dissolve completely. Before transformation, the re-dissolved bacterial solution is stayed on ice for over 10 minutes.

Based on the above, the recombinant vector, the transgenic fish using the same and the biomaterial using the same according to the invention can be applied to provide a transgenic fish that secrets the human procollagens or collagens from part(s) of the transgenic fish, so that part(s) of the transgenic fish that secrets the human procollagens or collagens can be treated as the biomaterials (e.g. the biomaterial having the human procollagens or collagens and the inorganic substances) and/or can be treated to purify the human procollagens or collagens.

While the disclosure has been described by the way of example and in terms of the preferred embodiments, it is to be understood that the invention need not be limited to the disclosed embodiments. On the contrary, it is intended to cover various modifications and similar arrangements included within the spirit and scope of the appended claims, the scope of which should be accorded the broadest interpretation to encompass all such modifications and similar structures.

What is claimed is:

1. A biomaterial comprising a recombinant human procollagen or collagen obtained from and secreted by a fish.

2. The biomaterial of claim 1 further comprising at least one fish scale of the fish, each having the recombinant human procollagen or collagen

3. The biomaterial of claim 1 wherein the recombinant human procollagen or collagen is expressed by at least one human procollagen or collagen gene in the fish.

4. A transgenic fish egg or embryo, comprising:
a fish chromosome; and
a recombinant vector, inserted in the fish chromosome, the recombinant vector having a human procollagen or collagen gene.

5. The recombinant fish egg or embryo of claim 4, wherein the recombinant vector further comprises an upstream gene, enabling expression of the human procollagen or collagen gene, and the human procollagen or collagen gene is connected to downstream of the upstream gene.

6. The transgenic fish egg or embryo of claim 5, wherein the upstream gene is a promoter existed in a fish, an enhancer existed in a fish, or combination thereof.

7. The transgenic fish egg or embryo of claim 5, wherein the recombinant vector further comprises a report gene, connected to downstream of the human procollagen or collagen gene.

8. The transgenic fish egg or embryo of claim 4, wherein the recombinant vector further comprises a report gene, connected to downstream of the human procollagen or collagen gene.

9. A recombinant vector comprising:
an upstream gene, constructed from a fish; and
a human procollagen or collagen gene, connected to downstream of the upstream gene.

10. The recombinant vector of claim 9, further comprising a report gene, connected to downstream of the human procollagen or collagen gene.

11. The recombinant vector of claim 9, wherein the upstream gene is a promoter existed in the fish, an enhancer existed in a fish, or combination thereof.

12. The biomaterial of claim 1, wherein each the human procollagen or collagen gene inserted in a fish chromosome of the fish.

13. The biomaterial of claim 2, wherein the recombinant human procollagen or collagen is secreted in the fish scale of the fish.

14. The biomaterial of claim 1, wherein the recombinant human procollagen or collagen is secreted in at least one fish scale of the fish.

15. The biomaterial of claim 1, wherein the recombinant human procollagen or collagen is secreted in at least one fish skins of the fish.

16. The biomaterial of claim 1, wherein the recombinant human procollagen or collagen is secreted in at least one fish eyes of the fish.

17. The biomaterial of claim 1, wherein the fish is a transgenic fish.


SEQUENCE LISTING (17 sequences)

SEQ ID NO: 1 - DNA, 1170 bp, Danio rerio
ggttcgacattggattttggtcgctttccaacacaacctaaatccaccaaatattaacttcctatgacatcgttattggacgtcaaaataacaatatccttagatgctggctagactttgaatttaggtcaccacaacctatatttaacctaatattaacatcttatgatgttgtgtgcctgctgggcaataactaaatgcactacagaatgttacgtttacacacatgtaaattacatgtaaatgcatcagcttttcacagcataatactcactacttactactcttgagtacttttaaaaaagctacttttcactcatactttgagtaatatttacaactgatacttttactcgcactacatttttaggcatgtattgatatttttactatgatttttcagtactctttccactactgcagccctccccatacataatcgtatgtttacacatatggtggagtttagagccataaactacattagctttgttagccgctagcattactgtgcagaattgtgtgtgtgcacattttccaatatcaatacagaaggaaactgtgttccctgttcccttgtaaatctcaacaatgcaactgttcagctcagggggaaaaatgccctgccagatccaaacgctggcaaaagtgaatggaaaaaagcctttcattaatgtgaaagttgctgcgcgccccacccagataaaaagagcagaggttaacatgctctctacggctgtccagccaaccagatactgaggcagaaacacacccgctggcagatggtgagagctacactgtctttttcagagtttctactggaatgcctgtcctcaagtctcaagcctctccttgcattttctcattccacctggggcaaagccccaggctgggtgtgacaacatttatcttaccactttctctctgtacctgtctaacaggtagggtgtgtgtgagagtgcgtatgtgtgcaagtgtgtgtgtgtgtgagagcagtcagctccaccctctcaagagtgtgtataaaattggtcagccagctgctgagagacacgcagagggactttgactctcctttgtgagcaacctcctccactcactcctctctcagagagcactctcgtacctccttctcagcaactcaaagacacaggcatcatgtcaaccagg

SEQ ID NO: 2 - DNA, 1570 bp, Danio rerio
gtcctgttttctagatcacaggtatttcaaagcatcattttcatcacaacaattatgcgtaattgtttttttaccattctcagatttcaaatcctgtgacattaaagctgggttcacaattttttttaacgtcatttataattgttccctgtcagacgttggacatgatcccagtgagatcttgggtaaaagccatgaagcagtgtgtgacatcaataatattttatgtcagacagtatcatcttgtcacacccaagactacagttccctttatggattttttcaaaatttgagcaaaattgttaccaaaagcaacagcacagtagctcagtggttagcactgttacgtcacagcaagaatgtcgctggtttgagtcccgactgtgctggtgtgggtttcctccgggtggtccggtttcccccacagtccaaatacatgcgctataggtgaattaggtaaactgaattggctgtagtgtatgtgtatgtgtgtgaatgcaagcatgtatgggtgtttcccaatactaggttggcaactggaagaccatccgctgtgtgaaacatacgccagaatagttggcggttcattccgctgtgacaacgttaaataagagactaagctgaagaaaaatgaacgaatgaatgctagcaaaatcataccatgtaaatctgggaagaacatggtcaagctattcctcagttactgaacttcaaaccatttgcttttatcaccttgtttttctatcttatcaacttgtttttctacaacttatcaatgttttcaaacacaacatatttgggcagttatgcaattttgagcaggcccttaaaaggtaaaaagcaagcgagcctatttgtagggctgcagtttgcagaaacatctcaaagaataaacacagagcttttagatattttaaagagatgctcttcaatcatgttgttgcaaacttcttcgtaagcttcttcaggggagttttgggtgaagtccccccttaagaatctgagttttgtaaacatgtttacatttaatccacatgtgtccactctttttggaaaatgcacatttaggtgtaacctgtcccaaaggtgcctgattgaaatcataaaccactgtttactttctcaaaccctgttcttctcagctaaacatgctatttaagttttcactgaagcattgtgggactgaaagcacaaaatgaggctgtatcttgttgcagtatgtatgtgtctagatgagcttggggattgggttcagatggagggagtcagaggggggagatcgaggcaggaaatgagaaagagagagaggatgtgggacgggcgttagagaacactgaaaaaagtaatgcaaaaggaaaaaaaccggaccatgtgggcagggtcttagttggtcagtgcgcgtgtgtgtgtttgagtcaaaaagagaaaaggaggaacagaaagagtttggtgggggtataaaagctgtacagttctcttctagtggctctcaagggttgacatccctcagcgcacactcatcttctactgcagccagagcttcattcctccaaaaccatgagcttctacg

SEQ ID NO: 3 - DNA, 4752 bp, Homo sapiens
agacgggagtttctcctcggggtcggagcaggaggcacgcggagtgtgaggccacgcatgagcggacgctaaccccctccccagccacaaagagtctacatgtctagggtctagacatgttcagctttgtggacctccggctcctgctcctcttagcggccaccgccctcctgacgcacggccaagaggaaggccaagtcgagggccaagacgaagacatcccaccaatcacctgcgtacagaacggcctcaggtaccatgaccgagacgtgtggaaacccgagccctgccggatctgcgtctgcgacaacggcaaggtgttgtgcgatgacgtgatctgtgacgagaccaagaactgccccggcgccgaagtccccgagggcgagtgctgtcccgtctgccccgacggctcagagtcacccaccgaccaagaaaccaccggcgtcgagggacccaagggagacactggcccccgaggcccaaggggacccgcaggcccccctggccgagatggcatccctggacagcctggacttcccggaccccccggaccccccggacctcccggaccccctggcctcggaggaaactttgctccccagctgtcttatggctatgatgagaaatcaaccggaggaatttccgtgcctggccccatgggtccctctggtcctcgtggtctccctggcccccctggtgcacctggtccccaaggcttccaaggtccccctggtgagcctggcgagcctggagcttcaggtcccatgggtccccgaggtcccccaggtccccctggaaagaatggagatgatggggaagctggaaaacctggtcgtcctggtgagcgtgggcctcctgggcctcagggtgctcgaggattgcccggaacagctggcctccctggaatgaagggacacagaggtttcagtggtttggatggtgccaagggagatgctggtcctgctggtcctaagggtgagcctggcagccctggtgaaaatggagctcctggtcagatgggcccccgtggcctgcctggtgagagaggtcgccctggagcccctggccctgctggtgctcgtggaaatgatggtgctactggtgctgccgggccccctggtcccaccggccccgctggtcctcctggcttccctggtgctgttggtgctaagggtgaagctggtccccaagggccccgaggctctgaaggtccccagggtgtgcgtggtgagcctggcccccctggccctgctggtgctgctggccctgctggaaaccctggtgctgatggacagcctggtgctaaaggtgccaatggtgctcctggtattgctggtgctcctggcttccctggtgcccgaggcccctctggaccccagggccccggcggccctcctggtcccaagggtaacagcggtgaacctggtgctcctggcagcaaaggagacactggtgctaagggagagcctggccctgttggtgttcaaggaccccctggccctgctggagaggaaggaaagcgaggagctcgaggtgaacccggacccactggcctgcccggaccccctggcgagcgtggtggacctggtagccgtggtttccctggcgcagatggtgttgctggtcccaagggtcccgctggtgaacgtggttctcctggccctgctggccccaaaggatctcctggtgaagctggtcgtcccggtgaagctggtctgcctggtgccaagggtctgactggaagccctggcagccctggtcctgatggcaaaactggcccccctggtcccgccggtcaagatggtcgccccggacccccaggcccacctggtgcccgtggtcaggctggtgtgatgggattccctggacctaaaggtgctgctggagagcccggcaaggctggagagcgaggtgttcccggaccccctggcgctgtcggtcctgctggcaaagatggagaggctggagctcagggaccccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggtccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgaccggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgctaaaggcgatgctggtccccctggccctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggcgagaaaggatcccctggtgctgatggtcctgctggtgctcctggtactcccgggcctcaaggtattgctggacagcgtggtgtggtcggcctgcctggtcagagaggagagagaggcttccctggtcttcctggcccctctggtgaacctggcaaacaaggtccctctggagcaagtggtgaacgtggtccccctggtcccatgggcccccctggattggctggaccccctggtgaatctggacgtgagggggctcctggtgccgaaggttcccctggacgagacggttctcctggcgccaagggtgaccgtggtgagaccggccccgctggaccccctggtgctcctggtgctcctggtgcccctggccccgttggccctgctggcaagagtggtgatcgtggtgagactggtcctgctggtcccgccggtcctgtcggccctgttggcgcccgtggccccgccggaccccaaggcccacgtggtgacaagggtgagacaggcgaacagggcgacagaggcataaagggtcaccgtggcttctctggcctccagggtccccctggccctcctggctctcctggtgaacaaggtccctctggagcctctggtcctgctggtccccgaggtccccctggctctgctggtgctcctggcaaagatggactcaacggtctccctggccccattgggccccctggtcctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggcagccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggccatccagctgaccttcctgcgcctgatgtccaccgaggcctcccagaacatcacctaccactgcaagaacagcgtggcctacatggaccagcagactggcaacctcaagaaggccctgctcctccagggctccaacgagatcgagatccgcgccgagggcaacagccgcttcacctacagcgtcactgtcgatggctgcacgagtcacaccggagcctggggcaagacagtgattgaatacaaaaccaccaagacctcccgcctgcgcatcatcgatgtggcccccttggacgttggtgccccagaccaggaattcggcttcgacgttggccatgtctgcttcctgtaaactccctccatcccaacctggctccctcccacccaaccaactttccccccaacccggaaacagacaagcaacccaaactgaaccccctcaaaagccaaaaaatgggagacaatttcacatggactttggaaaatatttttttcctttgcattcatctctcaaacttagtttttatctttgaccaaccgaacatgaccaaaaaccaaaagtgcattcaaccttaccaaaaaaaaaaaaaaaa

SEQ ID NO: 4 - DNA, 4572 bp, Homo sapiens
gtttcggctaagttggaggtactggccacgactgcatgcccgcgcccgccaggtgatacctccgccggtgacccaggggctctgcgacacaaggagtctgcatgtctaagtgctagacatgctcagctttgtggatacgcggactttgttgctgcttgcagtaaccttatgcctagcaacatgccaatctttacaagaggaaactgtaagaaagggcccagccggagatagaggaccacgtggagaaaggggtccaccaggccccccaggcagagatggtgaagatggtcccacaggccctcctggtccacctggtcctcctggcccccctggtctcggtgggaactttgctgctcagtatgatggaaaaggagttggacttggccctggaccaatgggcttaatgggacctagaggcccacctggtgcagctggagccccaggccctcaaggtttccaaggacctgctggtgagcctggtgaacctggtcaaactggtcctgcaggtgctcgtggtccagctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggacgtgttggtgcccctggcccagctggtgcccgtggcagtgatggaagtgtgggtcccgtgggtcctgctggtcccattgggtctgctggccctccaggcttcccaggtgcccctggccccaagggtgaaattggagctgttggtaacgctggtcctgctggtcccgccggtccccgtggtgaagtgggtcttccaggcctctccggccccgttggacctcctggtaatcctggagcaaacggccttactggtgccaagggtgctgctggccttcccggcgttgctggggctcccggcctccctggaccccgcggtattcctggccctgttggtgctgccggtgctactggtgccagaggacttgttggtgagcctggtccagctggctccaaaggagagagcggtaacaagggtgagcccggctctgctgggccccaaggtcctcctggtcccagtggtgaagaaggaaagagaggccctaatggggaagctggatctgccggccctccaggacctcctgggctgagaggtagtcctggttctcgtggtcttcctggagctgatggcagagctggcgtcatgggccctcctggtagtcgtggtgcaagtggccctgctggagtccgaggacctaatggagatgctggtcgccctggggagcctggtctcatgggacccagaggtcttcctggttcccctggaaatatcggccccgctggaaaagaaggtcctgtcggcctccctggcatcgacggcaggcctggcccaattggcccagctggagcaagaggagagcctggcaacattggattccctggacccaaaggccccactggtgatcctggcaaaaacggtgataaaggtcatgctggtcttgctggtgctcggggtgctccaggtcctgatggaaacaatggtgctcagggacctcctggaccacagggtgttcaaggtggaaaaggtgaacagggtcccgctggtcctccaggcttccagggtctgcctggcccctcaggtcccgctggtgaagttggcaaaccaggagaaaggggtctccatggtgagtttggtctccctggtcctgctggtccaagaggggaacgcggtcccccaggtgagagtggtgctgccggtcctactggtcctattggaagccgaggtccttctggacccccagggcctgatggaaacaagggtgaacctggtgtggttggtgctgtgggcactgctggtccatctggtcctagtggactcccaggagagaggggtgctgctggcatacctggaggcaagggagaaaagggtgaacctggtctcagaggtgaaattggtaaccctggcagagatggtgctcgtggtgctcctggtgctgtaggtgcccctggtcctgctggagccacaggtgaccggggcgaagctggggctgctggtcctgctggtcctgctggtcctcggggaagccctggtgaacgtggtgaggtcggtcctgctggccccaatggatttgctggtcctgctggtgctgctggtcaacctggtgctaaaggagaaagaggagccaaagggcctaagggtgaaaacggtgttgttggtcccacaggccccgttggagctgctggcccagctggtccaaatggtccccccggtcctgctggaagtcgtggtgatggaggcccccctggtatgactggtttccctggtgctgctggacggactggtcccccaggaccctctggtatttctggccctcctggtccccctggtcctgctgggaaagaagggcttcgtggtcctcgtggtgaccaaggtccagttggccgaactggagaagtaggtgcagttggtccccctggcttcgctggtgagaagggtccctctggagaggctggtactgctggacctcctggcactccaggtcctcagggtcttcttggtgctcctggtattctgggtctccctggctcgagaggtgaacgtggtctaccaggtgttgctggtgctgtgggtgaacctggtcctcttggcattgccggccctcctggggcccgtggtcctcctggtgctgtgggtagtcctggagtcaacggtgctcctggtgaagctggtcgtgatggcaaccctgggaacgatggtcccccaggtcgcgatggtcaacccggacacaagggagagcgcggttaccctggcaatattggtcccgttggtgctgcaggtgcacctggtcctcatggccccgtgggtcctgctggcaaacatggaaaccgtggtgaaactggtccttctggtcctgttggtcctgctggtgctgttggcccaagaggtcctagtggcccacaaggcattcgtggcgataagggagagcccggtgaaaaggggcccagaggtcttcctggcttaaagggacacaatggattgcaaggtctgcctggtatcgctggtcaccatggtgatcaaggtgctcctggctccgtgggtcctgctggtcctaggggccctgctggtccttctggccctgctggaaaagatggtcgcactggacatcctggtacagttggacctgctggcattcgaggccctcagggtcaccaaggccctgctggcccccctggtccccctggccctcctggacctccaggtgtaagcggtggtggttatgactttggttacgatggagacttctacagggctgaccagcctcgctcagcaccttctctcagacccaaggactatgaagttgatgctactctgaagtctctcaacaaccagattgagacccttcttactcctgaaggctctagaaagaacccagctcgcacatgccgtgacttgagactcagccacccagagtggagcagtggttactactggattgaccctaaccaaggatgcactatggatgctatcaaagtatactgtgatttctctactggcgaaacctgtatccgggcccaacctgaaaacatcccagccaagaactggtataggagctccaaggacaagaaacacgtctggctaggagaaactatcaatgctggcagccagtttgaatataatgtagaaggagtgacttccaaggaaatggctacccaacttgccttcatgcgcctgctggccaactatgcctctcagaacatcacctaccactgcaagaacagcattgcatacatggatgaggagactggcaacctgaaaaaggctgtcattctacagggctctaatgatgttgaacttgttgctgagggcaacagcaggttcacttacactgttcttgtagatggctgctctaaaaagacaaatgaatggggaaagacaatcattgaatacaaaacaaataagccatcacgcctgcccttccttgatattgcacctttggacatcggtggtgctgaccaggaattctttgtggacattggcccagtctgtttcaaataaatgaactcaatctaaattaaaaaagaaagaaatttgaaaaaactttctctttgccatttcttcttcttcttttttaactgaaagctgaatccttccatttcttctgcacatctacttgcttaaattgtgggcaaaagagaaaaagaaggattgatcagagcattgtgcaatacagtttcattaactccttccctcgctcccccaaaaatttgaatttttttttcaacactcttacacctgttatggaaaatgtcaacctttgtaagaaaaccaaaataaaaattgaaaaataaaaaccataaacatttgcaaaaaaaaaaaaaaaaaaaaaaaaaaaaaaaaaaaaaaaaaaa

SEQ ID NO: 5 - DNA, 4937 bp, Homo sapiens
ccatgattcgcctcggggctccccagacgctggtgctgctgacgctgctcgtcgccgctgtccttcggtgtcagggccaggatgtccaggaggctggcagctgtgtgcaggatgggcagaggtataatgataaggatgtgtggaagccggagccctgccggatctgtgtctgtgacactgggactgtcctctgcgacgacataatctgtgaagacgtgaaagactgcctcagccctgagatccccttcggagagtgctgccccatctgcccaactgacctcgccactgccagtgggcaaccaggaccaaagggacagaaaggagaacctggagacatcaaggatattgtaggacccaaaggacctcctgggcctcagggacctgcaggggaacaaggacccagaggggatcgtggtgacaaaggtgaaaaaggtgcccctggacctcgtggcagagatggagaacctgggacccctggaaatcctggcccccctggtcctcccggcccccctggtccccctggtcttggtggaaactttgctgcccagatggctggaggatttgatgaaaaggctggtggcgcccagttgggagtaatgcaaggaccaatgggccccatgggacctcgaggacctccaggccctgcaggtgctcctgggcctcaaggatttcaaggcaatcctggtgaacctggtgaacctggtgtctctggtcccatgggtccccgtggtcctcctggtccccctggaaagcctggtgatgatggtgaagctggaaaacctggaaaagctggtgaaaggggtccgcctggtcctcagggtgctcgtggtttcccaggaaccccaggccttcctggtgtcaaaggtcacagaggttatccaggcctggacggtgctaagggagaggcgggtgctcctggtgtgaagggtgagagtggttccccgggtgagaacggatctccgggcccaatgggtcctcgtggcctgcctggtgaaagaggacggactggccctgctggcgctgcgggtgcccgaggcaacgatggtcagccaggccccgcagggcctccgggtcctgtcggtcctgctggtggtcctggcttccctggtgctcctggagccaagggtgaagccggccccactggtgcccgtggtcctgaaggtgctcaaggtcctcgcggtgaacctggtactcctgggtcccctgggcctgctggtgcctccggtaaccctggaacagatggaattcctggagccaaaggatctgctggtgctcctggcattgctggtgctcctggcttccctgggccacggggccctcctggccctcaaggtgcaactggtcctctgggcccgaaaggtcagacgggtgaacctggtattgctggcttcaaaggtgaacaaggccccaagggagaacctggccctgctggcccccagggagcccctggacccgctggtgaagaaggcaagagaggtgcccgtggagagcctggtggcgttgggcccatcggtccccctggagaaagaggtgctcccggcaaccgcggtttcccaggtcaagatggtctggcaggtcccaagggagcccctggagagcgagggcccagtggtcttgctggccccaagggagccaacggtgaccctggccgtcctggagaacctggccttcctggagcccggggtctcactggccgccctggtgatgctggtcctcaaggcaaagttggcccttctggagcccctggtgaagatggtcgtcctggacctccaggtcctcagggggctcgtgggcagcctggtgtcatgggtttccctggccccaaaggtgccaacggtgagcctggcaaagctggtgagaagggactgcctggtgctcctggtctgaggggtcttcctggcaaagatggtgagacaggtgctgcaggaccccctggccctgctggacctgctggtgaacgaggcgagcagggtgctcctgggccatctgggttccagggacttcctggccctcctggtcccccaggtgaaggtggaaaaccaggtgaccagggtgttcccggtgaagctggagcccctggcctcgtgggtcccaggggtgaacgaggtttcccaggtgaacgtggctctcccggtgcccagggcctccagggtccccgtggcctccccggcactcctggcactgatggtcccaaaggtgcatctggcccagcaggcccccctggggctcagggccctccaggtcttcagggaatgcctggcgagaggggagcagctggtatcgctgggcccaaaggcgacaggggtgacgttggtgagaaaggccctgagggagcccctggaaaggatggtggacgaggcctgacaggtcccattggcccccctggcccagctggtgctaatggcgagaagggagaagttggacctcctggtcctgcaggaagtgctggtgctcgtggcgctccgggtgaacgtggagagactgggccccccggaccagcgggatttgctgggcctcctggtgctgatggccagcctggggccaagggtgagcaaggagaggccggccagaaaggcgatgctggtgcccctggtcctcagggcccctctggagcacctgggcctcagggtcctactggagtgactggtcctaaaggagcccgaggtgcccaaggccccccgggagccactggattccctggagctgctggccgcgttggacccccaggctccaatggcaaccctggaccccctggtccccctggtccttctggaaaagatggtcccaaaggtgctcgaggagacagcggcccccctggccgagctggtgaacccggcctccaaggtcctgctggaccccctggcgagaagggagagcctggagatgacggtccctctggtgccgaaggtccaccaggtccccagggtctggctggtcagagaggcatcgtcggtctgcctgggcaacgtggtgagagaggattccctggcttgcctggcccgtcgggtgagcccggcaagcagggtgctcctggagcatctggagacagaggtcctcctggccccgtgggtcctcctggcctgacgggtcctgcaggtgaacctggacgagagggaagccccggtgctgatggcccccctggcagagatggcgctgctggagtcaagggtgatcgtggtgagactggtgctgtgggagctcctggagcccctgggccccctggctcccctggccccgctggtccaactggcaagcaaggagacagaggagaagctggtgcacaaggccccatgggaccctcaggaccagctggagcccggggaatccagggtcctcaaggccccagaggtgacaaaggagaggctggagagcctggcgagagaggcctgaagggacaccgtggcttcactggtctgcagggtctgcccggccctcctggtccttctggagaccaaggtgcttctggtcctgctggtccttctggccctagaggtcctcctggccccgtcggtccctctggcaaagatggtgctaatggaatccctggccccattgggcctcctggtccccgtggacgatcaggcgaaaccggccctgctggtcctcctggaaatcctggaccccctggtcctccaggtccccctggccctggcatcgacatgtccgcctttgctggcttaggcccgagagagaagggccccgaccccctgcagtacatgcgggccgaccaggcagccggtggcctgagacagcatgacgccgaggtggatgccacactcaagtccctcaacaaccagattgagagcatccgcagccccgagggctcccgcaagaaccctgctcgcacctgcagagacctgaaactctgccaccctgagtggaagagtggagactactggattgaccccaaccaaggctgcaccttggacgccatgaaggttttctgcaacatggagactggcgagacttgcgtctaccccaatccagcaaacgttcccaagaagaactggtggagcagcaagagcaaggagaagaaacacatctggtttggagaaaccatcaatggtggcttccatttcagctatggagatgacaatctggctcccaacactgccaacgtccagatgaccttcctacgcctgctgtccacggaaggctcccagaacatcacctaccactgcaagaacagcattgcctatctggacgaagcagctggcaacctcaagaaggccctgctcatccagggctccaatgacgtggagatccgggcagagggcaatagcaggttcacgtacactgccctgaaggatggctgcacgaaacataccggtaagtggggcaagactgttatcgagtaccggtcacagaagacctcacgcctccccatcattgacattgcacccatggacataggagggcccgagcaggaattcggtgtggacatagggccggtctgcttcttgtaaaaacctgaacccagaaacaacacaatccgttgcaaacccaaaggacccaagtactttccaatctcagtcactctaggactctgcactgaatggctgacctgacctgatgtccattcatcccaccctctcacagttcggacttttctcccctctctttctaagagacctgaactgggcagactgcaaaataaaatctcggtgttctatttatttattgtcttcctgtaagaccttcgggtcaaggcagaggcaggaaactaactggtgtgagtcaaatgccccctgagtgactgcccccagcccaggccagaagacctcccttcaggtgccgggcgcaggaactgtgtgtgtcctacacaatggtgctattctgtgtcaaacacctctgtattttttaaaacatcaattgatattaaaaatgaaaagattattggaaagtaaaaaaaaaaaaaaaaaaaaaaaaaaaaaaaa

SEQ ID NO: 6 - DNA, 28 bp, Artificial Sequence
  forward primer for amplification of keratin 4 promoter with engineered ApaI site
gggcccggttcgacattggattttggtc

SEQ ID NO: 7 - DNA, 28 bp, Artificial Sequence
  reverse primer for amplification of keratin 4 promoter with engineered BamHI site
ggatccgatgcctgtgtctttgagttgc

SEQ ID NO: 8 - DNA, 33 bp, Artificial Sequence
  forward primer for amplification of collagen type 1 alpha 1 with engineered AgeI site
accggtaccatgttcagctttgtggacctccgg

SEQ ID NO: 9 - DNA, 29 bp, Artificial Sequence
  reverse primer for amplification of collagen type 1 alpha 1 with engineered SalI site
gtcgacttacaggaagcagacatggccaa

SEQ ID NO: 10 - DNA, 37 bp, Artificial Sequence
  forward primer for amplification of collagen type 1 alpha 1 with engineered Bgl2 and Nhe1 sites
agatctgctagcaccatgttcagctttgtggacctcc

SEQ ID NO: 11 - DNA, 27 bp, Artificial Sequence
  reverse primer for amplification of collagen type 1 alpha 1 with engineered Sal1 site
gtcgacttacaggaagcagacatggcc

SEQ ID NO: 12 - DNA, 37 bp, Artificial Sequence
  forward primer for amplification of collagen type 1 alpha 2 with engineered BamH1 and Nhe1 sites
ggatccgctagcaccatgctcagctttgtggatacgc

SEQ ID NO: 13 - DNA, 33 bp, Artificial Sequence
  reverse primer for amplification of collagen type 1 alpha 2 with engineered Sal1 and Cla1 sites
gtcgacatcgatttatttgaaacagactgggcc

SEQ ID NO: 14 - DNA, 27 bp, Artificial Sequence
  forward primer for amplification of collagen type 2 alpha 1 with engineered EcoRI site
gaattcatgattcgcctcggggctccc

SEQ ID NO: 15 - DNA, 27 bp, Artificial Sequence
  reverse primer for amplification of collagen type 2 alpha 1 with engineered SolI site
gtcgacttacaagaagcagaccggccc

SEQ ID NO: 16 - DNA, 35 bp, Artificial Sequence
  forward primer for amplification of collagen type 2 alpha 1 with engineered BamH1 and Nhe1 sites
ggatccgctagcaccatgattcgcctcggggctcc

SEQ ID NO: 17 - DNA, 33 bp, Artificial Sequence
  reverse primer for amplification of collagen type 2 alpha 1 with engineered Sal1 and Cla1 sites
gtcgacatcgatttacaagaagcagaccggccc